(19) United States
(12) Patent Application Publication
(10) Pub. No.: US 2023/0137702 A1
(43) Pub. Date: May 4, 2023

(54) DIGITIZER CALIBRATION CHECK

(71) Applicant: THINK SURGICAL, INC., Fremont, CA (US)

(72) Inventors: Steve Henderson, Fremont, CA (US); Daniel P. Bonny, Fremont, CA (US)

(73) Assignee: THINK SURGICAL, INC., Fremont, CA (US)

(21) Appl. No.: 17/917,954

(22) PCT Filed: Apr. 8, 2021
(86) PCT No.: PCT/US2021/026350

§ 371 Date: Oct. 10, 2022

Related U.S. Application Data
(60) Provisional application No. 63/006,765, filed on Apr. 8, 2020.

(51) Int. Cl. A61B 34/20; A61B 90/00

(52) U.S. Cl. CPC A61B 34/20; A61B 90/39; A61B 2034/2046

(57) ABSTRACT

A method for verifying the calibration of a digitizer during a computer-assisted medical procedure is provided utilizing a tracked digitizer and a secondary tracking array (e.g., a bone tracking array). A medical system for performing the computerized method for verifying the calibration of a digitizer during a computer-assisted medical procedure is provided. A method for verifying the calibration of a tracking array relative to a feature with the system includes a first calibration definition and a second calibration definition transmitted to the tracking system. A first feature and a second feature together are assembled. The calibration is verified by computing the deviations between the tracked position of the first feature and the tracked position of the second feature using: a recorded position and orientation of the first and second tracking array, and the uploaded first calibration definition and the uploaded second calibration definition.

### RELATED APPLICATIONS

This application claims priority benefit of U.S. Provisional Application Ser. No. 63/006,765 filed 8 Apr. 2020; the content of which is hereby incorporated by reference.

### TECHNICAL FIELD

The present invention generally relates to computer-assisted medical procedures, and more particularly to a system and method for checking the calibration of a digitizer to ensure the digitizer functions accurately during a computer-assisted medical procedure.

### BACKGROUND

Throughout a lifetime, bones and joints become damaged and worn through normal use, disease, and traumatic events. Arthritis is a leading cause of joint damage that leads to cartilage degradation, pain, stiffness, and bone loss overtime. Arthritis can also cause the muscles articulating the joints to lose strength and become very painful.

If the pain associated with the dysfunctional joint is not alleviated by less-invasive therapies, a joint arthroplasty procedure is considered as a treatment. Joint arthroplasty is an orthopedic procedure in which an arthritic or dysfunctional joint surface is replaced with an orthopedic prosthesis.

The accurate placement and alignment of an implant is a large factor in determining the success of joint arthroplasty. A slight misalignment may result in poor wear characteristics, reduced functionality, poor clinical outcomes, and decreased prosthetic longevity.

Computer-assisted orthopedic surgery is an expanding field having applications in total joint arthroplasty (TJA), bone fracture repair, maxillofacial reconstruction, and spinal reconstruction. For example, the TSOLUTION ONE® Surgical System (THINK Surgical, Inc., Fremont, Calif.) aids in the planning and execution of total hip arthroplasty (THA) and total knee arthroplasty (TKA). The TSOLUTION ONE® Surgical System includes: a pre-operative planning software program to generate a surgical plan using an image data set of the patient's bone and computer-aided design (CAD) files of several implants; and an autonomous surgical robot that precisely mills the bone to receive an implant according to the surgical plan.

In order to achieve accurate implant placement and alignment, a surgical plan is typically generated using 3-D bone models of the patient's bones and one or more implant models of a desired implant. A user positions the implant models relative to the bone models to designate the best fit, fill, and/or alignment of the implants on the bones. The plan is then transferred to a robotic-assisted surgical device in the operating room (OR) to accurately execute the plan.

For any robotic-assisted surgical device, the bone's position and orientation (POSE) needs to be known relative to the device and the surgical plan to accurately create the cuts in bone according to the plan. The bone's POSE relative to the device and plan may be initially determined using a process called registration. Several registration procedures are known in the art, illustratively including pin-based, point-to-point, point-to-surface, laser scanning, image-free, and image registration, as described in U.S. Pat. Nos. 5,951,475; 6,033,415; 8,287,522; and 8,010,177. After registration, the bone needs to be tracked to update the bone's POSE in real-time relative to the surgical device as there is bone movement as cutting the adjustment of tension on the bone occurs.

Conventional tracking systems include optical tracking systems, electromagnetic tracking systems, and mechanical tracking systems. Each of these tracking prior art systems requires a tracking reference device fixed to the patient's bone prior to registration to provide a link for the tracking system to track the bone. Then after registration, the prior art tracking system can accurately track the bone in real-time. For example, an optical tracking system utilizes a tracking array fixed to the patient's bone, an electromagnetic tracking system, utilizes a magnetic field transmitter fixed to the bone, and a mechanical tracking system utilizes a distal end of one or more articulating linkages fixed to the bone.

A digitizer is a tool used during computer-assisted surgery to primarily assist in the collection of points on a bone. The collected points are used to register the bone to imaging data and/or surgical planning data relative to a fixed coordinate reference frame.

FIG. 1 illustrates an example of a prior art tracked digitizer 10. The tracked digitizer generally includes a shaft 12 having a digitizing tip 14 at one end, and a tracking array 16 either assembled or integrated with an opposing end of the shaft 12. The tracking array 16 may include three or more fiducial markers (18a, 18b, and 18c) to permit a tracking system (e.g., an optical tracking system) to track the digitizer 10 in three dimensions.

In order to accurately collect points on the bone with a tracked digitizer 10, the geometric relationship between the tracking array 16 and the digitizer tip 14 needs to be defined. In other words, the position of the digitizer tip 14 needs to be known relative to the tracked POSE of the tracking array 16, such that the position of the digitizer tip 14 can be accurately tracked by the tracking system. The geometric relationship may be defined or established based on the known geometric relationship between the digitizer tip and to the tracking array when the digitizer is manufactured (e.g., the tip 14 is positioned 20 mm from the center of the tracking array 16). This is only applicable when the tracking array 16 is an integral part of the digitizer 10. Due to manufacturing errors, and in cases where the tracking array 16 is a separate part that is assembled with the other components to form the digitizer, the digitizer 10 is calibrated using techniques known in the art to accurately define the geometric relationship. It is of note that at least the tip 14 must be amendable to sterilization. In the operating room (OR), the calibration of the digitizer 10 is checked to ensure the tracking system is accurately tracking the digitizer tip 14 since the digitizer may have been bent or damaged before use. It is desirable that the intra-operative calibration check of the digitizer be as efficient as possible from both a time and accuracy perspective and the conventional devices and methods of use could be more efficient.

Thus, there exists a need to efficiently check the calibration of a tracked digitizer to ensure the digitizer tip is accurately tracked during a computer-assisted medical procedure to address at least some of the aforementioned issues with the prior art.

### SUMMARY OF THE INVENTION

A system for verifying the calibration of a tracking member relative to a feature is provided. The system includes a first tracking member with a first feature and a second tracking member with a second feature. A first calibration definition of the first feature and a second calibration definition of the second feature is stored on a tracking system. The tracking includes a processor and software executable instructions that when executed by the processor computes the deviations between the tracked position of the first feature and the tracked position of the second feature when assembled together using: a) a recorded position and orientation (POSE) of the first tracking member and the second tracking member when the first feature is assembled to the second feature; and b) the uploaded first calibration definition and the uploaded second calibration definition. The tracking system verifies the calibration when the computed deviations between the first feature and second feature are within pre-defined acceptable criteria.

A method for verifying the calibration of a digitizer during a computer-assisted medical procedure is provided utilizing a digitizer and a bone tracking array. The digitizer has a digitizer tip, a digitizer tracking array, and a stored calibration definition of the tip position, and the bone tracking array having a stored calibration definition of the feature position. The stored calibration definition of the tip position is transmitted to a tracking system in an operating room where the computer-assisted medical procedure is taking place. The stored calibration definition of the feature position is transmitted to the tracking system. The position and orientation (POSE) of the digitizer tracking array and the bone tracking array when the digitizer tip and feature are assembled together are recorded with the tracking system. The deviation between the tracked position of the digitizer tip and the tracked position of the feature is recorded using: a) the recorded POSE of the digitizer tracking array and the bone tracking array; and b) the transmitted calibration definition of the tip position and the transmitted calibration definition of the feature position. The calibration is accepted if the deviation is within pre-defined acceptable criteria.

A method for verifying the calibration of a digitizer during a computer-assisted surgical procedure is provided that includes a bone tracking array being attached to a bone subject to the surgical procedure. A position of a digitizer tip at a distal end of a digitizer is calibrated relative to a digitizer tracking array mounted at a proximal end of the digitizer. The digitizer tip is placed in physical contact with a feature on the bone tracking array. The digitizer is pivoted around in space while the digitizer tip remains in the feature. A center of rotation of the digitizer is calculated, where the center of rotation is indicative of the position of the digitizer tip. The calculated center of rotation is compared with the calibrated position of the digitizer, where if the comparison is in agreement, then the calibration check is accepted.

A medical system for performing the computerized method for checking and verifying the calibration of a digitizer during a computer-assisted medical procedure is provided. The system includes a surgical robot or hand-held surgical device with an end effector tool. A workstation includes a computer, user-peripherals, and a monitor for displaying a graphical user interface (GUI). At least one of a mechanical digitizer or a non-mechanical tracking system is provided. The computer also includes a processor, non-transient storage memory, and other hardware, software, data and utilities to execute the method. The user peripherals allow a user to interact with the GUI and include user input mechanisms including at least one of a keyboard, mouse, controller, joystick, foot pedal, pendant, digitizer, or a monitor with touchscreen capabilities.

A method for verifying the calibration of a tracking array relative to a feature with the aforementioned system includes a first calibration definition and a second calibration definition to being uploaded to the tracking system. A first feature and a second feature together are assembled. The calibration is verified by computing, with the tracking system, the deviations between the tracked position of the first feature and the tracked position of the second feature using: a) a recorded position and orientation (POSE) of the first tracking array and the second tracking array; and b) the uploaded first calibration definition and the uploaded second calibration definition.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 is a schematic of an prior art tracked digitizer;

FIG. 2 is a flowchart of a method for checking the calibration of a digitizer in accordance with an embodiment of the invention;

FIG. 3 illustrates a system for implementing the method for checking calibration of a digitizer in accordance with certain embodiments of the invention;

FIG. 4 is a flowchart of a method for checking the calibration of a digitizer in accordance with an embodiment of the invention;

FIG. 5 is a top view of a bone tracking array for use in implementing the method of FIG. 4 in accordance with an embodiment of the invention;

FIG. 6 is a top view of a digitizer for use in implementing the method of FIG. 4 in accordance with an embodiment of the invention;

FIG. 7 is a perspective view of a handheld surgical tool suitable for use in embodiments of the inventive calibration method;

FIG. 8 depicts a surgical system in the context of an operating room (OR) with a hand-held surgical device, where the surgical system is capable of performing embodiments of the inventive method for checking calibration of a digitizer during a robotic-assisted orthopedic surgery; and

FIGS. 9A and 9B are detailed views of the hand-held surgical device of FIG. 8 in first and second working position and orientation (POSE), respectively.

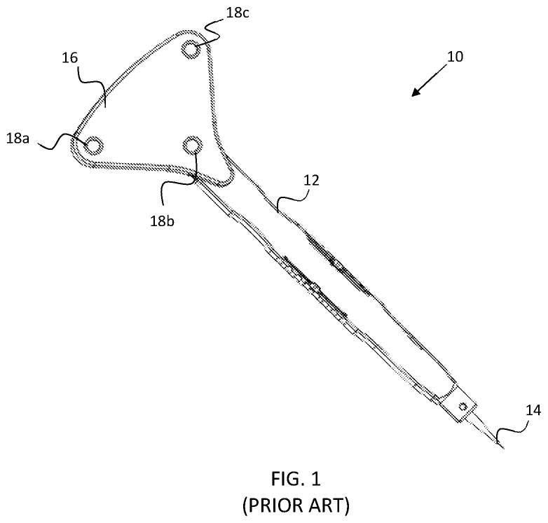

### DETAILED DESCRIPTION

The present invention has utility as a system and method to efficiently check the calibration of a digitizer to ensure the digitizer tip is accurately tracked during a robotic procedure as exemplified by a computer-assisted surgical procedure. The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Patent documents and publications mentioned in the specification are indicative of the levels of those skilled in the art to which the invention pertains. These documents and publications are incorporated herein by reference to the same extent as if each individual document or publication was specifically and individually incorporated herein by reference in their entirety.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

As used herein, the term “pre-procedure data” refers to data used to plan a medical procedure prior to making modifications to tissue. The pre-procedure data may include one or more of the following: an image data set of tissue (e.g., an image data set acquired via computed tomography (CT), magnetic resonance imaging (MRI), ultrasound, x-ray, laser scan, etc.), a virtual generic model of the tissue, a physical model of the tissue, a virtual patient-specific model of the tissue generated from an image data set of the tissue, a set of data collected directly on the tissue intra-operatively (commonly used with imageless computer-assist devices), etc. As used herein, the term “pre-operative bone data” refers to pre-procedure data involving a bone.

As used herein, the term “digitizer” refers to a device capable of measuring, collecting, recording, and/or designating the location of physical locations (e.g., points, lines, planes, boundaries, etc.) or tissue structures in three-dimensional space. By way of example but not limitation, the “digitizer” may be: a “mechanical digitizer” having passive links and joints, such as the high-resolution electro-mechanical sensor arm described in U.S. Pat. No. 6,033,415 (which U.S. patent is hereby incorporated herein by reference); a non-mechanically tracked digitizer probe (e.g., optically tracked, electromagnetically tracked, acoustically tracked, and equivalents thereof) as described for example in U.S. Pat. No. 7,043,961 (which U.S. patent is hereby incorporated herein by reference); an end-effector of a robotic device; or a laser scanner.

As used herein, the term “digitizing” refers to the collecting, measuring, and/or recording of physical locations or tissue structures in space with a digitizer.

Also described herein are “computer-assisted medical systems.” A computer assisted medical system refers to any system requiring a computer to aid in a medical procedure. Examples of computer-assisted medical systems include 1-N degree of freedom hand-held surgical systems, tracking systems, tracked passive instruments, active or semi-active hand-held surgical devices and systems, autonomous serial-chain manipulator systems, haptic serial chain manipulator systems, parallel robotic systems, or master-slave robotic systems, as described in U.S. Pat. Nos. 5,086,401; 7,206,626; 8,876,830; 8,961,536; and 9,707,043; and the robotic surgical system described in U.S. patent application Ser. No. 16/875,173. In particular inventive embodiments, the computer-assisted medical system is a computer-assisted surgical system such as a robotic surgical system as described below. In particular inventive embodiments, the surgical system is a 2-DOF articulating device as described in U.S. Patent Publication 2018/0344409. The surgical system may provide autonomous, semi-autonomous, or haptic control and any combinations thereof. In addition, a user may manually maneuver a tool attached to the surgical system while the system provides at least one of power, active, or haptic control to the tool.

As used herein, the term “real-time” refers to the processing of input data within milliseconds such that calculated values are available within 2 seconds of computational initiation

As used herein, the term “registration” refers to: the determination of the spatial relationship between two or more objects; the determining of a coordinate transformation between two or more coordinate systems associated with those objects; and/or the mapping of an object onto another object. Examples of objects routinely registered in an operating room (OR) illustratively include: computer-assisted systems/devices; anatomy (e.g., bone); pre-procedure data (e.g., 3-D virtual bone models); medical planning data (e.g., an implant model positioned relative to pre-operative bone data, a cut-file defined relative to an implant model and/or pre-operative bone data, virtual boundaries defined relative to an implant model and/or pre-operative bone data, virtual planes defined relative to an implant model and/or pre-operative bone data, or other cutting parameters associated with or defined relative to an implant model and/or the pre-operative bone data); and any external landmarks (e.g., a tracking array affixed to a bone, an anatomical landmark, a designated point/feature on a bone, etc.) associated with the tissue (if such landmarks exist). Methods of registration known in the art are described in U.S. Pat. Nos. 6,033,415; 8,010,177; 8,036,441; and 8,287,522; and U.S. Patent Application Publication 2016/0338776, which patents and publications are hereby incorporated herein by reference. In particular embodiments with orthopedic procedures, the registration procedure relies on the manual collection of several points (i.e., point-to-point, point-to-surface) on the bone using a tracked digitizer where the surgeon is prompted to collect several points on the bone that are readily mapped to corresponding points or surfaces on a representation of the bone (e.g., a 3-D bone model). The points collected from the surface of a bone with the digitizer may be matched using iterative closest point (ICP) algorithms to generate a transformation matrix. The transformation matrix provides the correspondence between the position of the bone in an operating room (OR) with the bone model to permit the surgical device to execute the plan.

Also used herein is the term “optical communication” which refers to wireless data transfer via infrared or visible light that are described in U.S. Pat. No. 10,507,063 and assigned to the assignee of the present application.

Embodiments of the present invention describe a system and method to efficiently check the calibration of a digitizer to ensure that a tip of the digitizer is accurately tracked by a tracking system. While the present invention is further detailed with respect to a TKA procedure in the accompanying drawings, it is to be understood that the present invention is applicable to computer-assisted medical procedures in general and regardless of anatomy, as well as manufacturing processes. By way of example but not limitation, the system and method of the present invention may be applicable to medical procedures performed on: a) hard tissues (e.g., bones, teeth) including bones in the hip, ankle, shoulder, spine, jaw, skull, elbow, wrist, hands, fingers, feet, toes, etc., as well as revision of initial repair or replacement of any joints or bones; and b) soft tissues (e.g., organs, muscles, connective tissue) including the brain, ligaments, tendons, lungs, heart, skin, etc. Exemplary manufacturing processes that benefit from the present invention include composite material part adhesive bead line application and cutting of composite materials. Composite materials are routinely used in the aerospace, vehicle, and sporting goods manufacturing sectors.

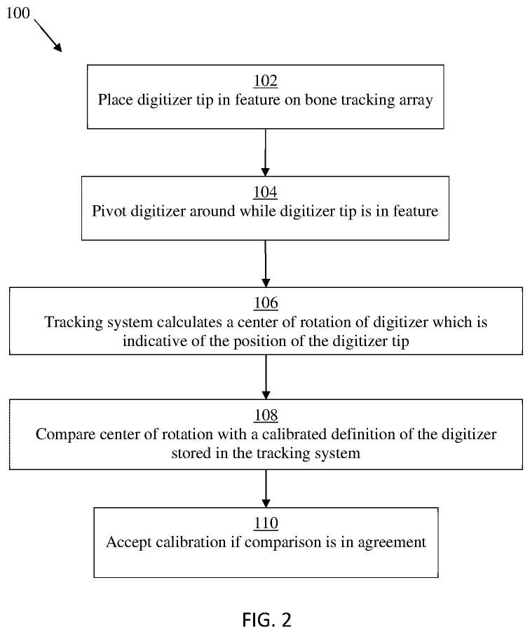

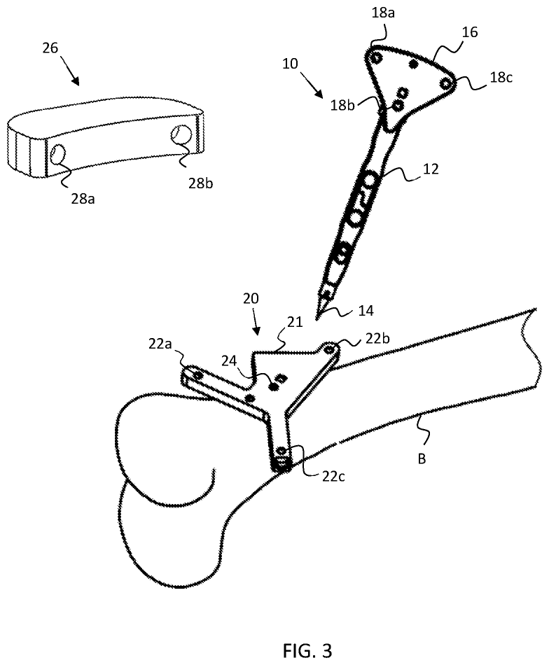

With reference now to the figures, FIG. 2 illustrates a particular embodiment of a method 100 for checking the calibration of a digitizer, and FIG. 3 depicts the components thereof. The components may include a digitizer 10, a bone tracking array 20, and a tracking system 26. The digitizer 10 may include a shaft 12 having a digitizer tip 14 at one end, and a digitizer tracking array 16 integrated or assembled to an opposing end of the shaft 12. The position of the digitizer tip 14 is calibrated relative to the digitizer tracking array 16, where this calibration is referred to herein as the calibration definition of the digitizer 10. The calibration definition is stored in the tracking system 26 computer and/or another computer in data transfer communication with the tracking system 26. It should be appreciated that the calibration definition may be defined and stored as a point, an axis, an axis and a point, and/or a transformation. The bone tracking array 20 is a tracking array that is fixable to a bone B. The bone tracking array 20 may be a rigid body 21 having at least three fiducial markers (22a, 22b, 22c) arranged about the rigid body 21, and at least one feature 24 formed on the rigid body 21. The feature 24 may be for example a divot, a hole, a recession, or other feature capable of receiving the digitizer tip 14 into physical contact therewith. The tracking system 26 may be an optical tracking system having two or more cameras (28a, 28b) to track the tracking arrays (16, 20) in three-dimensional space. It is appreciated that the tracking system in specific embodiments may be an electromagnetic tracking system or a mechanical tracking system. During the workflow of a medical procedure, a computer-assisted medical system may prompt the user to check the calibration of the digitizer 10 prior to using the digitizer 10 for its intended purpose (e.g., collecting points for registration). In some inventive embodiments, the feature 24 has a non-symmetric shape that is complementary to the cross-section of the tip 14 to allow the insertion of the tip 14 into the feature in only a single orientation with a tab on the tip that allows for rotation within a rotary slot complementary to the tab after non-degenerate insertion of the tip into the feature.

Continuing with FIG. 2, embodiments of the inventive method may include placing the digitizer tip 14 in the feature 24 on the bone tracking array 20 (Block 102) and pivoting the digitizer 10 around in space while the digitizer tip 14 remains in the feature 24 (Block 104). The tracking system 26 calculates a center of rotation of the digitizer 10, where the center of rotation is indicative of the position of the digitizer tip 14 (Block 106). The center of rotation may be calculated by fitting a circle or sphere to the tracked positions of the digitizer tracking array 16 created while pivoting the digitizer 10. The calculated center of rotation is then compared with the calibrated definition of the digitizer 10 stored in the tracking system 22 (Block 108). If the comparison is in agreement therewith, then the calibration check is accepted, meaning the tracked position of the digitizer tip 14 is considered accurate relative to the digitizer tracking array 16 (Block 110). The comparison may be in agreement if there is an exact match between the calibrated tip position and the calculated center of rotation, or if there is a statistical match between the two (e.g., within a standard deviation). If the comparison is not in agreement, then the calibration check is rejected, meaning the calibrated tip position is not accurate. Failure of the calibration check may result in the user being prompted to repeat the calibration check or to get a new digitizer for use during the medical procedure.

In some inventive embodiments, the tracking system 26 filters the data when computing the center of rotation to ensure the data is not poorly weighted due to the user's input (i.e., the input being the pivoting of the digitizer 10 in the feature 24). In other inventive embodiments, the tracking system 26 detects the motion of the digitizer 10 and determines whether the range of the user's input was adequate. In other inventive embodiments, the tracking system 26 and/or another component of the computer-assisted medical system provides guidance for the medical workflow and the exceptions when the calibration check fails.

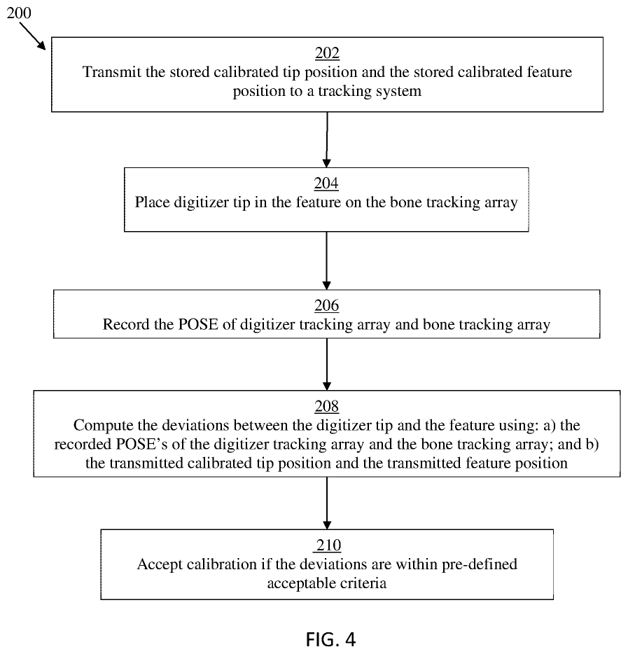

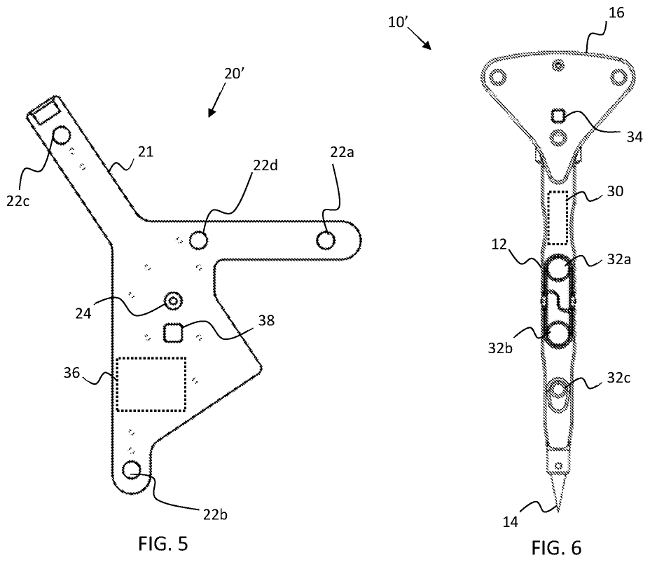

With reference now to FIGS. 4-6, a particular inventive embodiment of a method 200 to check the calibration is depicted, where FIG. 4 depicts the steps of the method, while FIGS. 5 and 6 depict some of the inventive components thereof. The components may include a digitizer 10′, a bone tracking array 20′, and a tracking system 26 (as shown in FIG. 3). The digitizer 10′ may include a shaft 12 having a digitizer tip 14 at one end, and a digitizer tracking array 16 integrated or assembled to an opposing end of the shaft 12. The digitizer 10′ may further include computing components 30, input mechanisms 32, and a transmitter 34 to optically communicate data to the tracking system 26. The computing components 30 may be computing components well known in the art, for example, a processor, microprocessor, microcontroller, memory (e.g., RAM, ROM, flash, secondary memory), circuitry, a power source, etc. The computing components 30 may further include software components, where the software components may include, for example, data, utilities, and software executable instructions. The input mechanisms 32 and transmitter 34 are in communication with the computing components 30. The input mechanisms 32 may include one or more buttons (32a, 32b), switches 32c, voice commands, or other input mechanisms that allow the user to provide input to the medical system by way of the digitizer 10′. The digitizer 10′ may further include a feedback signaling mechanism such as a blinking light to provide feedback to the user. The transmitter 34 optically transmits data between the digitizer 10′ and the tracking system 26 or other components of the computer-assisted medical system as described in U.S. Pat. No. 10,507,063. The transmitter 34 may be a light emitting diode that is modulated to transmit data via visible or infrared light, however, other types of transmitters may be used to for other types of data transfer mechanisms (e.g., radio-frequency, WiFi, Bluetooth). The bone tracking array 20′ is a tracking array that is fixable to a bone B. The bone tracking array 20′ may be a rigid body 21 having at least three fiducial markers (22a, 22b, 22c, 22d) arranged about the rigid body 21, and at least one feature 24 formed on the rigid body 21. The feature 24 may be for example, a divot, a hole, a recession, or other feature capable of receiving or restraining the digitizer tip 14. The bone tracking array 20′ may further include computing components 36 and a transmitter 38. The computing components 36 may be computing components well known in the art, for example, a processor, microprocessor, microcontroller, memory (e.g., RAM, ROM, flash, secondary memory), circuitry, a power source, etc. The computing components 36 may further include software components, where the software components may include, for example, data, utilities, and software executable instructions. The transmitter 38 optically transmits data between the bone tracking array 20 and the tracking system 26 or other components of the computer-assisted medical system as described in U.S. Pat. No. 10,507,063. The transmitter 34 may be a light emitting diode that is modulated to transmit data via visible or infrared light, however, other types of transmitters may be used for other types of data transfer mechanisms (e.g., radio-frequency, WiFi, Bluetooth).

A method 200 for checking the calibration of the digitizer 10′ using the components of FIGS. 5 and 6 is outlined in FIG. 4. The method 200 greatly reduces the amount of time it will take to develop the digitizer calibration check workflow, make the setup easier for the user, and mitigate some risks relative to the aforementioned prior art. The method 200 also does not require the filtering of data for weighting, or the possibility of having an inadequate range of motion of the aforementioned method 100. The method 200 may include the digitizer 10′ and the bone tracking array 20′. The digitizer 10′ may have a calibration definition of the tip position stored in the digitizer computing components 30, where the calibration definition of the tip position defines the position of the digitizer tip 14 relative to the digitizer tracking array 16 (or more specifically, relative to the fiducial markers 18 of the digitizer tracking array 16). The bone tracking array 20′ may have a calibration definition of the feature position stored in the bone tracking array computing components 36, where the calibration definition of the feature position defines the position of the feature 24 relative to the bone tracking array fiducial markers 22. The method 200 may include the following. In the operating room, the user may be prompted to verify the calibration of the digitizer 10′ prior to using the digitizer 10′ for its intended use. To verify the calibration, the stored calibration definition of the tip 14 position and the stored calibration definition of the feature 24 position are transmitted to the tracking system 26 [Block 202]. The transmission (e.g., the downloading or uploading of data) may occur via wireless or wired communication, and in particular embodiments the transmission is accomplished via optical communication using the transmitters 34 and 38, respectively, in the operating room. A user, prior to or after the transmission of the calibration definitions, assembles the digitizer tip 14 with the feature 24 on the bone tracking array 20 (Block 204), and the tracking system 26 records the position and orientation (POSE) of the digitizer tracking array 16 and the bone tracking array 20′ [Block 206]. The deviations between the position of the digitizer tip 14 and the position of the feature 24 (when assembled) is computed using: a) the recorded POSE of the digitizer tracking array 16 and the bone tracking array 20′; and the transmitted calibration definition of the tip position and the transmitted calibration definition of the feature position [Block 208]. The calibration is verified/accepted if the deviations are within pre-defined acceptable criteria [Block 210], meaning the position of the digitizer tip 14 is being accurately tracked relative to the digitizer tracking array 16. If the deviations are outside of the pre-defined acceptable criteria, then the calibration check is rejected, and the user is prompted to repeat the calibration check or get a new digitizer 10′. Specific embodiments of the method 200 is described below.

The position of the digitizer tip 14 and the position of the feature 24 are initially calibrated prior to the use of the device (e.g., before entering the operating room, before the device is opened from its packaging, or before verifying the calibration as described herein). The calibration of the digitizer tip 14 may be performed using calibration techniques well known in the art such as those described in U.S. Pat. Nos. 10,792,109 and 7,043,961, which results in a calibration definition of the position of the digitizer tip 14 relative to the digitizer tracking array 16 that is stored in the computing components 30 of the digitizer 10′. The stored calibration definition of the tip position may be stored as a mathematical or geometric expression such as a point (or coordinate (x, y, z)), an axis (or vector \vec{V}), an axis and a point, or a transformation (i.e., a transformation matrix or a component thereof) that defines the position of the digitizer tip 14 relative to the digitizer tracking array 16. For example, the calibration definition of the tip position may be defined as: (i) a point that relates the tip position to the digitizer tracking array 14; (ii) an axis that relates the axis of the shaft 12 to the digitizer tracking array 14; (iii) an axis and a point that relates the axis of the shaft 12 and the coordinates of the tip position relative to the digitizer tracking array 16; or (iv) a full or partial transformation matrix that can be applied to the tracked position of the digitizer tracking array 16 to determine the tip position. As for the feature 24 on the bone tracking array 20′, the position of the feature 24 may be calibrated using techniques known in the art, or the techniques further described below, which results in a calibration definition of the position of the feature 24 relative to the bone tracking array fiducial markers 22 that is then stored in the computing components 26 of the bone tracking array 20′. The calibration definition of the feature position may be stored as a mathematical or geometric expression such as a point (or coordinate (x, y, z), an axis (or vector \vec{V}), an axis and a point, or a transformation (i.e., a transformation matrix or a component thereof) that defines the position of the feature 24 relative to the bone tracking array fiducial markers 22. For example, the calibration definition of the feature position may be defined as: (i) a point (or coordinates) that relate the position of the feature 24 to the fiducial markers 22; (ii) an axis that relates an axis that is normal to the plane of the bone tracking array 20′ and originates at or intersects through the feature 24 to the bone tracking array fiducial markers 22; or (iii) a full or partial transformation matrix that can be applied to the tracked position of the digitizer tracking array 16 to determine the feature position.

The position of the feature 24 on the bone tracking array 20 may be calibrated (i.e., the position of the feature 24 on the bone tracking array 20 is defined and/or determined relative to the positions of the fiducial markers (22a, 22b, 22c)) using techniques known in the art (e.g., utilizing coordinate-measuring-machines). In a particular embodiment, the position of the feature 24 is calibrated using the method steps Block 102 to Block 106 in the method 100 shown in FIG. 2, but in a controlled environment with proper manufacturing controls and acceptance testing. For example, in the controlled environment (e.g., a factory), a technician or computer-controlled device may place a tracked digitizer 10 in the feature 24, pivot the digitizer 10 while tracking the position of the digitizer 10, and calculate the center of rotation, where the center of rotation defines the position of the feature 24 on the bone tracking array 20. The position of the feature 24 is then stored in the bone tracking array 20 as the calibration definition of the feature position. In some inventive embodiments in which an extraordinary accuracy of the calibrated position of the feature 24 is required using this method, the following aspects may be considered. First, a dedicated standard calibrated digitizer, which may be created and shaped specifically for this calibration, is used under a controlled environment. A first assumption may be that both the concerned bone tracking array and the standard calibrated digitizer are optimally tracked (e.g., an optimal optical angle of tracking). The position of the feature 24 on the bone tracking array 20 may be chosen with this optimally tracked consideration. A second assumption may be that the calibration of the feature 24 is obtained with the usual circular (or spherical) fitting method with the calibrated standard digitizer and the process may be as long as needed with motions on multiple axes of an optimal jitter correction. With these considerations, the obtained final position of the feature 24 (relative to the fiducial markers (22a, 22b, 22c) on the bone tracking array 20) is exceptionally accurate. Here again, the use of a rotationally non-degenerate match between the feature and the tip cross-section is readily invoked. Once the calibrated position of the feature 24 is defined or obtained, this calibration definition may be stored in the computing components 36 of the bone tracking array 20 during manufacturing or post-manufacturing (possibly as the translation component of the calibration transformation matrix, which for a bone tracking array 20 is not normally used for anything).

In particular inventive embodiments, there may not be a need for calibrating the position of the feature 24 using the method steps of Blocks 102 to 106. The manufacturing of the bone tracking array 20 may already be within well-defined and narrow manufacturing tolerances, such that the position of the feature 24 is accurate enough when manufactured. An occasional test on specific production batches could check that the tolerances are within range. Even a rough approximation of the position of the feature 24 that considers the manufacturing tolerances may be accurate enough, where the approximated position of the feature 24 can be stored and recorded in the bone tracking array 20. As an additional safety step, a user may need to pivot the digitizer 10 in at least one specific complete circular motion (at least one axis) for a few seconds to safely guarantee a rejected calibration check when the exact position of the feature 24 is not calibrated.

The method 200 also includes recording the POSE of the digitizer tracking array 16 and the bone tracking array 20′ when the digitizer tip 14 and feature 24 are assembled together. In particular embodiments, the feature 24 is a divot where the user places the digitizer tip 14 in the divot to assemble the digitizer tip 14 with the feature 24. The tracking system 26 then records the POSE of the digitizer tracking array 16 and the bone tracking array 20′. The tracking system 26 (or a computer in communication with the tracking system 26) computes the deviations between the position of the digitizer tip 14 and the feature 24 using: a) the recorded POSE of the digitizer tracking array 16 and the bone tracking array 20′; and b) the transmitted calibration definition of the tip position and the transmitted calibration definition of the feature position. In a specific embodiment, the tracking system 26 calculates the deviation in the following manner. The tracking system 26 calculates the position of the digitizer tip 14 in space by applying the transmitted calibration definition of the tip position (e.g., a transformation matrix) to the recorded POSE of the digitizer tracking. In other words, the position of the digitizer tip 14 as tracked by the tracking system 26 is calculated by transforming the recorded POSE of the digitizer tracking array 16 by the calibration definition of the tip position. The tracking system 26 likewise calculates the position of the feature 24 in space by applying the transmitted calibration definition of the feature position with the recorded POSE of the digitizer tracking array 20′. In other words, the position of the feature 24 as tracked by the tracking system is calculated by transforming the recorded POSE of the bone tracking array 20′ by the calibration definition of the feature position. The difference between the calculated position of the digitizer tip 14 and the feature 24 is the deviation. Since the calibrated position of the feature 24 is known with a high degree of accuracy as described above, any deviation suggests that the digitizer tip 14 is not being accurately tracked (i.e., the calibrated definition of the tip position is not accurate). In such a case, the user is prompted to repeat the calibration check or obtain a new digitizer for use. If the deviation is within pre-defined acceptable criteria, then the calibration is verified. The pre-defined acceptable criteria may be chosen according to the application. For example, in computer-assisted surgical procedures, there is a need for a high degree of accuracy, where the pre-defined acceptable criteria may require at least one of the following: an exact match; 0.01-0.1 mm (millimeter) deviation; 0.01-0.5 mm deviation; 0.01-1 mm deviation; 0.01-2 mm deviation; 0.01-5 mm deviation; or 0.01-10 mm deviation. However, the pre-defined acceptable criteria can vary based on the needs and accuracy requirements, so as long as there is a pre-defined acceptable criteria established, the calibration can be verified accordingly.

Another particular inventive embodiment of a system and method for checking the calibration of one or more devices is also described herein. The system may generally include a first tracking member (e.g., a first tracking array) with a first feature, a second tracking member (e.g., a second tracking array) with a second feature, a first calibration definition of the first feature, a second calibration definition of the second feature, where the first calibration definition and the second calibration definition are stored by a tracking system 26. The first calibration definition and the second calibration definition may be pre-stored on the tracking system 26 prior to the procedure, or the calibration definitions may be transmitted (e.g., uploaded) to the tracking system 26 in the OR (by way of the tracking members) and then stored on the tracking system. To check the calibration of the first feature and/or the second feature, a user assembles the first feature and the second feature together, where the tracking system computes the deviations between the first feature and the second feature using: a) the tracked POSEs of the first tracking member and the second tracking member; and b) the first calibration definition and the second calibration definition. Specific embodiments of the system and method are further described below.

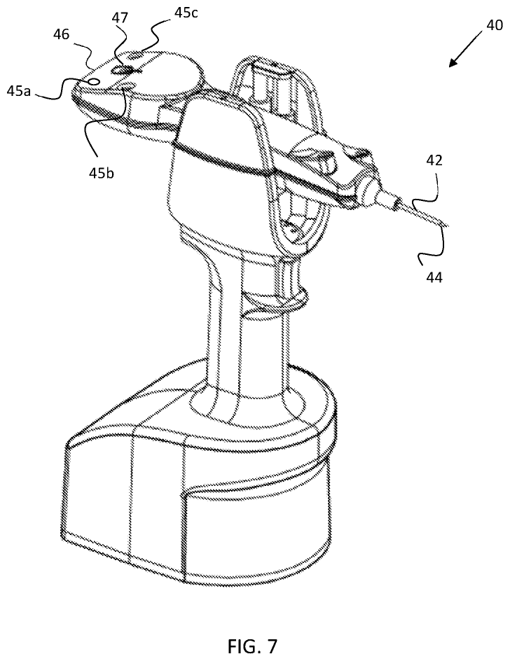

The first tracking member and second tracking member are the elements that a tracking system identifies to track an object. For example, the tracking members may be tracking arrays (e.g., tracking arrays (16, 20)) of an optical tracking system, electromagnetic sensors of an electromagnetic tracking system, a distal end of an electro-mechanical tracking system, transponders of a radio-frequency location system, and the like. In particular embodiments, the first tracking member is a bone tracking array 24 having a first feature. The second tracking is the digitizer tracking array 16, where the second feature is a shaft 12, or digitizer tip, of a digitizer 10. In a particular embodiment, with reference to FIG. 7, a 2-DoF surgical device 40 is shown that operates a tool 42 having a tool tip 44 and a device tracking array 46. Here, the second feature may be a shaft of the tool 42, or the shaft of an end-effector operated by a robotic surgical system. The first calibration definition and the second calibration definition may be stored in computing components of the first tracking array and/or second tracking array, respectively. The first calibration definition and second calibration definition may be defined and/or stored as a point, an axis, an axis and a point, or a transformation. For example, the calibration definition may be an axis that may define: (i) an axis of the shaft 12 of the digitizer 10 relative to the fiducial markers (18a, 18b, 18c) on the digitizer tracking array 16; or (ii) an axis of the feature 24 relative to the fiducial markers (22a, 22b, 22c) on the bone tracking array 20. The calibration definition may further be an axis and/or a point, where the axis and/or point may define for example: (i) the axis of the shaft 12 of the digitizer 10 and the point of the digitizer tip 16 relative to the fiducial markers (18a, 18b, 18c) on the digitizer tracking array 16; (ii) the point (i.e., position/coordinates) of the feature 24 relative to the fiducial markers (22a, 22b, 22c) on the bone tracking array 20; or (iii) an axis and point of the feature 24 relative to the fiducial markers (22a, 22b, 22c) on the bone tracking array 20. The calibration definition may further be a full or partial transformation (i.e., a transformation matrix) that may define for example: (i) the position of the digitizer tip 14 relative to the fiducial markers (18a, 18b, 18c) on the digitizer tracking array 16; or (ii) the position of the feature 24 on the bone tracking array 20 relative to its fiducial markers (22a, 22b, 22c) on the bone tracking array 20.

The deviations between the first feature and the second feature may be computed in the same manner as described above, where the tracking system 26 applies the first calibration definition to the recorded POSE of the first tracking array to determine the tracked position of the first feature, and applies the second calibration definition to the recorded POSE of the second tracking array to determine the tracked position of the second feature. Any difference between the determined tracked position of the first feature and second feature is the deviation. If the deviation is within pre-defined acceptable criteria, then the calibration of the first feature and/or second feature is verified. If the deviation is outside or does not coincide with the pre-defined acceptable criteria, then the user is prompted as such and instructed to proceed accordingly.

It is further contemplated, that the first feature and/or second feature need not be fixedly attached to another object. For example, it is not necessary for a bone tracking array 20′ to be fixedly attached to a bone to verify the calibration of a digitizer 10′. Both the first feature and second feature can be floating in space, and as long as the tracking system can track and record the first tracking array and the second tracking array while the first feature and second feature are assembled together, then the verification of the calibration can occur in the same manner as described herein.

Furthermore, it should be appreciated that the first tracking member or second tracking member may be any tracking member that the tracking system tracks. For example, the first tracking member may be coupled to a surgical device that operates a first tool as described above. Then, the second tracking member may be a calibration tracking member dedicated only for the purpose of verifying the calibration of the first tool. Or, the second tracking member may be coupled or integrated with a second tool, where the second tracking member is still used to verify the calibration of the first tool.

### Surgical System

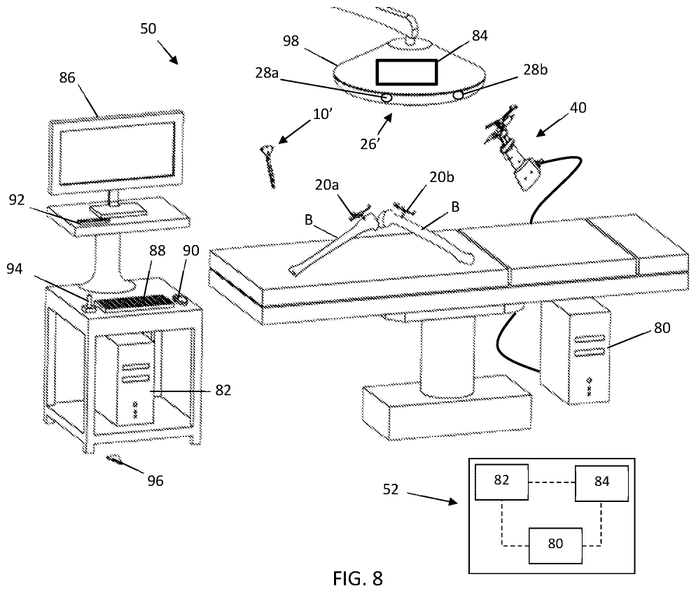

FIG. 8 is a schematic view showing an example of a computer-assisted medical system being a computer-assisted surgical system 50 for performing computer-assisted surgery. The surgical system 50 includes a 2-DoF articulating surgical device 40 (referred to hereinafter as 2-DoF device), a computing system 52, and a tracking system 26.

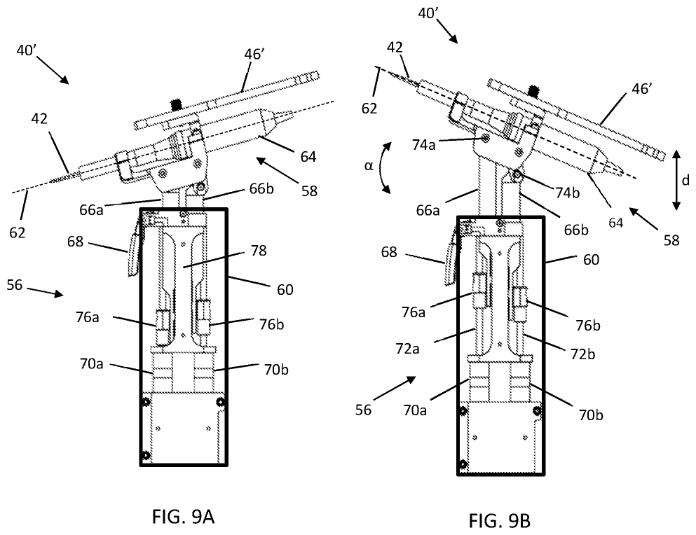

FIGS. 9A and 9B are schematic views showing a particular 2-DoF device 40′ of a surgical system 50 of FIG. 8 in greater detail. More particularly, FIG. 9A shows the 2-DoF device 40′ in a first working POSE, and FIG. 9B illustrates the 2-DoF device 40′ in a second working POSE. The 2-DoF device 40′ has a hand-held portion 56 and a working portion 58. The hand-held portion 56 has an outer casing 60 of ergonomic design which can be held and wielded by a user (e.g., a surgeon). The working portion 58 has a tool 42 (e.g., drill bit, burr, removable bone pin) having a tool axis 62. The tool 42 is readily attached to the working portion 58 and driven by a motor 64. The hand-held portion 56 and working portion 58 are connected to one another by a front linear rail 66a and a back linear rail 66b that are actuated by components in the hand-held portion 56 in order to control the pitch and translation of the working portion 58 relative to the hand-held portion 56, as will hereinafter be discussed in further detail. A device tracking array 46, having three or more fiducial markers (45a, 45b, 45c) (FIG. 7) of the sort well known in the art, is preferably rigidly attached to the working portion 58 in order to permit the tracking system 26′ (which may be mounted in surgical light 98, on a boom, or on a wall of the OR) (FIG. 8) to track the POSE of the working portion 58. The three or more fiducial markers (45a, 45b, 45c) may, alternatively, be integrated directly onto the working portion 58 as shown in FIG. 7. The fiducial markers (45a, 45b, 45c) may be active markers such as light emitting diodes (LEDs), or passive markers such as retroreflective spheres. The 2-DoF device 40′ may further include one or more user input mechanisms such as a triggers (e.g., trigger 68) or button(s). The user input mechanisms may permit the user to perform various functions illustratively including: activating or deactivating the motor 64; activating or deactivating the actuation of the working portion 204; notifying the computing system 52 to change from targeting one virtual plane to a subsequent virtual plane; and pausing the surgical procedure.

Continuing with FIGS. 9A and 9B, within the outer casing of the hand-held portion 56 are a front actuator 70a that powers a front ball screw 72a (or lead screw), and a back actuator 70b that powers a back ball screw 72b (or lead screw). The actuators (front actuator 70a, back actuator 70b) are preferably servo-motors that bi-directionally rotate the ball screws (72a, 72b). A first end of the linear rails (front linear rail 66a, back linear rail 66b) are attached to the working portion 58 via hinges (74a, 74b), such that the hinges (74a, 74b) allow the working portion 58 to pivot relative to the linear rails (66a, 66b,). Ball nuts (76a, 76b) (or lead nuts) are attached at a second end of the linear rails (66a, 66b). The ball nuts (76a, 76b) are in mechanical communication with the ball screws (72a, 72b). The actuators (70a, 70b) power the ball screws (72a, 72b) which in turn cause the ball nuts (76a, 76b) to translate along the axis of the ball screws (72a, 72b). Translation of ball nuts (76a, 76b) along ball screws (72a, 72b), respectively, causes translation of front linear rail 66a and back linear rail 66b, respectively, to permit (a) selective linear movement of working portion 58 relative to hand-held portion 56, and (b) selective pivoting of working portion 58 relative to hand-held portion 56 of the 2-DoF device 40′. Accordingly, the translation “d” and pitch “a” (FIG. 9B) of the working portion 58 may be adjusted depending on the position of each ball nut (76a, 76b) on their corresponding ball screw (72a, 72b). A linear guide 78 (FIG. 9A) may further constrain and guide the motion of the linear rails (208a, 208b) in the translational direction “d”.

The 2-DoF device 40′ may receive power via an input/output port (e.g., from an external power source) and/or from on-board batteries (not shown).

The actuators (70a, 70b) and motor 64 of the 2-DoF device 40′ may be controlled using a variety of methods. By way of example but not limitation, according to one method of the present invention, control signals may be provided via an electrical connection to an input/output port. By way of further example but not limitation, according to another method of the present invention, control signals are communicated to the 2-DoF device 40′ via a wireless connection, thereby eliminating the need for electrical wiring. In a specific embodiment, the wireless connection may be made via optical communication, where the 2-DoF device 40′ includes a transmitting LED 47. In a particular embodiment, the 2-DoF device 40′ includes a receiver for receiving control signals from the computing system 52. The receiver may be, for example, an input port for a wired connection (e.g., Ethernet port, serial port), a transmitter, a modem, a wireless receiver (e.g., Wi-Fi receiver, Bluetooth® receiver, a radiofrequency receiver, an optical receiver (e.g., photosensor, photodiode, camera)), or a combination thereof. The receiver may send control signals from the computing system 52 directly to the actuators (70a, 70b) and/or motor 64 of the 2-DoF device 40′, or the receiver may be in communication with a processor (e.g., an on-board device computer 80 as further described below) to pre-process the control signals before sending to the actuators (70a, 70b) and/or motor 64.

Referring again to FIG. 8, the computing system 52 generally includes hardware and software for executing a surgical procedure. By way of example but not limitation, in one inventive embodiment of the present invention, the computing system 52 is configured to maintain the bone pin axis 62 (FIG. 9B) coincident with a virtual plane defined in a surgical plan independent of the POSE of the hand-held portion 56. The computing system 52 accurately maintains the bone pin axis 62 coincident with a virtual plane based on the registered and tracked POSE of the virtual plane relative to the tracked POSE of the working portion 58. While the computing is depicted as a unit positioned in proximity to the other inventive components, it is appreciated that the computing system 52 need not be unitary or within the confines of the operating room. Computational efforts is readily distributed among other computers or even completed within the cloud.

The computing system 52 may include: a device computer 80 (or controller) including a processor; a planning computer 82 (or controller) including a processor; a tracking computer 84 (or controller) including a processor; and peripheral devices. Processors operate in the computing system 52 to perform computations and execute software associated with the inventive system and method. The device computer 80, the planning computer 82, and the tracking computer 84 may be separate entities as shown in FIG. 8, or it is also contemplated that operations may be executed on one (or two) computers depending on the configuration of the surgical system 50. For example, the tracking computer 84 may have operational data to control the 2-DoF device 40 without the need for a device computer 80. Alternatively, if desired, the device computer 80 may include operational data to plan the surgical procedure without the need for the planning computer 82. Furthermore, if desired, any combination of the device computer 80, planning computer 82, and/or tracking computer 84 may be connected together via a wired or wireless connection. In addition, the data gathered by, and/or the operations performed by, the tracking computer 84 and/or device computer 80 may work together to control the 2-DoF device 40 and, as such, the data gathered by, and/or the operations performed by, the tracking computer 84 and/or device computer 80 to control the 2-DoF device 40 may be referred to herein as a “control system”.

The peripheral devices allow a user to interface with the computing system 52 and may include, but are not limited to, one or more of the following: one or more user-interfaces, such as a display or monitor 86 to display a graphical user interface (GUI); and user-input mechanisms, such as a keyboard 88, mouse 90, pendent 92, joystick 94, and foot pedal 96. If desired, the monitor 86 may have touchscreen capabilities, and/or the digitizer (10, 10′) and/or the 2-DoF device 40 may include one or more input mechanisms (e.g., voice commands, triggers, buttons, switches, etc.) to interface with the computing system 52.

The device computer 80 may include one or more processors, controllers, software, data, utilities, and/or storage medium(s) (e.g., RAM, ROM or other non-volatile or volatile memory) to perform functions related to the operation of the 2-DoF device 40. By way of example but not limitation, the device computer 80 may include software, data, and utilities to control the POSE of the working portion 58, receive and process tracking data, control the speed of the motor 64, execute registration algorithms, execute calibration routines, provide workflow instructions to the user throughout a surgical procedure, as well as any other suitable software, data or utilities required to successfully perform the procedure in accordance with embodiments of the invention. The device computer 80 may be located separate from the 2-DoF device 40 as shown in FIG. 8, or the device computer 80 may be housed in the hand-held portion 56 of the 2-DoF device 40 to provide on-board control. If the device computer 80 is housed in the 2-DoF device 40, referred to hereinafter as on-board device computer, the on-board device computer may receive external data (e.g., tracking data, informational data, workflow data, etc.) via a wired or wireless connection. Similarly, an on-board device computer may send internal data (e.g., operational data, actuator/ball-screw position data, battery life, etc.) via a wired or wireless connection. In a preferred embodiment, external data may be received and/or internal data is sent wirelessly using optical communications. Details about bi-directional optical communication between a 2-DoF device 40 and a tracking system 26 is further described below.

The planning computer 82 is preferably dedicated to planning the procedure either pre-operatively or intra-operatively. By way of example but not limitation, the planning computer 82 may contain hardware (e.g., processors, controllers, memory, etc.), software, data, and utilities capable of: receiving and reading medical imaging data; segmenting imaging data; constructing and manipulating three-dimensional (3D) virtual models; storing and providing computer-aided design (CAD) files such as implant CAD files, bone pin CAD files; planning the POSE of implants, bone tunnels, and/or 3-D virtual ligament or tendon grafts relative to the bone; generating the surgical plan data for use with the system 50; and/or providing other various functions to aid a user in planning the surgical procedure. The final surgical plan data may include pre-procedure data (e.g., an image data set of the bone), bone registration data, subject identification information, and/or the POSE of one or more implants, virtual boundaries, virtual axes, virtual planes, cut-files (e.g., cutting instructions/parameters), soft tissue boundaries, targeted soft tissues, etc. defined relative to the desired tissue. The device computer 80 and the planning computer 82 may be directly connected in the operating room, or may exist as separate entities in different locations. The final surgical plan is readily transferred to the device computer 80 and/or tracking computer 84 through a wired (e.g., electrical connection) or a wireless connection (e.g., optical communication) in the operating room (OR); or transferred via a non-transient data storage medium (e.g., a compact disc (CD), or a portable universal serial bus (USB drive)) if the planning computer 82 is located outside the OR. As described above, the computing system 52 may include one or more computers, with multiple processors capable of performing the functions of the device computer 80, the tracking computer 84, the planning computer 82, or any combination thereof.

The tracking system 26′ (FIG. 8) of the present invention generally includes a detection device to determine the POSE of an object relative to the position of the detection device. In particular embodiments of the present invention, the tracking system 26′ is an optical tracking system such as the optical tracking system described in U.S. Pat. No. 6,061,644; and having two or more optical cameras (28a, 28b) for detecting the position of fiducial markers arranged on rigid bodies or integrated directly on the tracked object. By way of example but not limitation, the fiducial markers may include: an active transmitter, such as an LED or electromagnetic radiation emitter; a passive reflector, such as a plastic sphere with a retro-reflective film; or a distinct pattern or sequence of shapes, lines or other characters. A set of fiducial markers arranged on a rigid body is sometimes referred to herein as a tracking array, wherein each tracking array includes a unique geometry/arrangement of fiducial markers, or a unique transmitting wavelength/frequency (if the markers are active LEDS), such that the tracking system 26′ can distinguish between each of the tracked objects.

In specific inventive embodiments, the tracking system 26′ may be incorporated into a surgical light 98 (FIG. 8), located on a boom, a stand, or built into the walls or ceilings of the operating room. The tracking system computer 84 includes tracking hardware, software, data, and/or utilities to determine the POSE of objects (e.g., bones such as the femur F and tibia T, the 2-DoF device 40) in a local or global coordinate frame. The output from the tracking system 26′ (i.e., the POSE of the objects in 3-D space) is referred to herein as tracking data, where this tracking data is readily communicated to the device computer 80 through a wired or wireless connection. In a specific embodiment, the tracking computer 26′ processes the tracking data and provides control signals directly to the 2-DoF device 40 and/or device computer 80 based on the processed tracking data to control the position of the working portion 58 of the 2-DoF device 40 relative to the hand-held portion 56.

The tracking data is preferably determined using the position of the fiducial markers detected from the optical cameras (28a, 28b) and operations/processes such as image processing, image filtering, triangulation algorithms, geometric relationship processing, registration algorithms, calibration algorithms, and coordinate transformation processing.

The tracking system 26′ may further receive and store data related to the calibration definitions described herein, and perform the comparisons/calculations for the calibration checks of a device (e.g., check the calibration of a digitizer (10, 10′) or a 2-DoF device 40 with the methods described herein). The tracking system 26′ may further include a transmitter for transmitting data to the 2-DoF device 40.

Bi-directional optical communication may occur between the 2-DoF device 40 and the tracking system 26′ by way of a modulated light source (e.g., light emitting diode (LED)) and a photosensor (e.g., photodiode, camera). The 2-DoF device 40 may include an LED 47 and a photosensor (i.e., a receiver) disposed on the working portion 58 or hand-held portion 56, where the LED and photosensor are in communication with at least one of a modem, a processor, or an on-board device computer. Data generated internally by the 2-DoF device 40 may be sent to the tracking system 26′ by modulating the LED, where the light signals (e.g., infrared, visible light) created by the modulation of the LED are detected by the tracking system optical detectors (e.g., cameras) or a dedicated photosensor and processed by the tracking system computer 84. The tracking system 106 may likewise send data to the 2-DoF device 40 with a modulated LED associated with the tracking system 26′. Data generated by the tracking system 26′ may be sent to the 2-DoF device 40 by modulating the LED on the tracking system 26′, where the light signals are detected by the photosensor on the 2-DoF device 40 and processed by a processor in the 2-DoF device 40. Examples of data sent from the tracking system 26′ to the 2-DoF device 40 includes operational data, medical planning data, informational data, control data, positional or tracking data, pre-procedure data, or instructional data. Examples of data sent from the 2-DoF device 40 to the tracking system 26′ may include motor position data, battery life, operating status, logged data, operating parameters, warnings, or faults.

### OTHER EMBODIMENTS

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A calibration system, comprising:
a first device comprising:
a first body;
a plurality of first fiducial markers coupled to the first body at a plurality of fixed first marker locations; and
a first feature coupled to the first body at a first feature location wherein the first feature location has an expected location with respect to the plurality of fixed first marker locations;
a second device comprising:
a second body;
a plurality of second fiducial markers coupled to the second body at a plurality of fixed second marker locations;
a second feature positioned on the second body at a fixed feature location;
a data storage component storing the fixed second marker locations and the fixed feature location; and
a transmitter for transmitting signals indicating the fixed second marker locations and the fixed feature location;
a tracking system for locating the plurality of first fiducial markers and the plurality of second fiducial markers;
a receiver for receiving signals from the transmitter indicating the fixed second marker locations and the fixed feature location; and
a computer operatively coupled to the receiver, the computer comprising a processor configured to determine if the expected location of the first feature matches the location of the second feature within predefined acceptable criteria when the first feature is coupled to the second feature.

2-4. (canceled)

5. The system of claim 1 wherein the second feature is at least one of a divot, a hole, or a recession.

6. The system of claim 1 wherein the second device is configured to be affixed to a bone.

7. The system of claim 1 wherein the first feature is an end or tip of at least one of a shaft, a tool, or an instrument.

8. The system of claim 7 wherein the first feature is a distal tip of a digitizer.

9. The system of claim 1 wherein the first device is a surgical device.

10. The system of claim 9 wherein the surgical device is at least one of a pin driver, an articulating hand-held device, a serial-chain manipulator robot device, or a stewart-gough robotic device.

11. (canceled)

12. The system of claim 1 wherein the first device comprises a second data storage component storing the plurality of first marker locations and the first feature location.

13. (canceled)

14. (canceled)

15. A method for verifying the calibration of a first device with the system of claim 1, the method comprising:
transmitting the signals indicating the fixed second marker locations and the fixed feature location to the receiver; and
determining, with the computer, if the expected location of the first feature matches the location of the second feature within predefined acceptable criteria when the first feature is coupled to the second feature.

16-34. (canceled)

35. The system of claim 1 wherein the expected location of the first feature location is defined by a first calibration definition that defines the first feature location with respect to the plurality of fixed first marker locations.

36. The system of claim 1 wherein the data storage component stores the fixed feature location as a second calibration definition that defines the fixed feature location with respect to the plurality of fixed second marker locations.

37. The system of claim 1 wherein each fiducial marker of the plurality of first fiducial markers and the plurality of second fiducial markers is at least one of: an active transmitter; an electromagnetic sensor; or a passive reflector.

38. The system of claim 1 wherein the data storage component is non-volatile memory.

39. A tracking device, comprising:
a body;
a plurality of fiducial markers coupled to the body at a plurality of fixed marker locations;
a feature positioned on the body at a fixed feature location;
a data storage component storing the fixed marker locations and the fixed feature location; and
a transmitter for transmitting the fixed marker locations and the fixed feature location to a receiver.

40. The tracking device of claim 40 wherein the data storage component stores the fixed feature location as a calibration definition that defines the fixed feature location with respect to the plurality of fixed marker locations.

41. The tracking device of claim 40 wherein the data storage component is non-volatile memory.

42. The tracking device of claim 40 wherein the feature is at least one of a divot, a hole, or a recession.

43. The tracking device of claim 40 wherein the body is configured to be affixed to a bone.

44. The tracking device of claim 40 wherein each fiducial marker of the plurality of fiducial markers is at least one of: an active transmitter; an electromagnetic sensor; or a passive reflector.

45. The tracking device of claim 40 wherein the transmitter is a light emitting diode (LED).
